(19) United States
(12) Patent Application Publication
(10) Pub. No.: US 2011/0271892 A1
(43) Pub. Date: Nov. 10, 2011

(54) REPOSITORY FOR BIO-MASS FARMING SYSTEM

(75) Inventors: Minoo Homi Patel, Billericay Essex (GB); Feargal Brennan, London (GB); Naresh Magan, Hertfordshire (GB)

(73) Assignee: Cranfield University, Cranfield, Bedfordshire (GB)

(21) Appl. No.: 13/139,722

(22) PCT Filed: Nov. 30, 2009
(86) PCT No.: PCT/GB2009/051617

§ 371 Date: Jul. 28, 2011

(30) Foreign Application Priority Data
Dec. 15, 2008 (GB) 08228082

(51) Int. Cl. C12M 1/02; B63B 21/00

(52) U.S. Cl. 114/230.2; 435/289.1

(57) ABSTRACT

A repository (3) for the water-based fanning of bio-mass, wherein the repository is configured to be attachable to at least one other repository at six regularly-spaced locations (7) on its periphery.

### TECHNICAL FIELD

The present invention relates to bio-mass farming systems, in particular the production of bio-mass including algae strains on oceans or lake surfaces.

### BACKGROUND ART

It is an established fact that burning fossil fuels is steadily increasing the concentration of carbon dioxide (CO₂) in the atmosphere. There is increasingly stronger evidence to indicate that these CO₂ levels are causing global temperatures to rise and leading to the so called climate change phenomenon which may have serious consequences in the long run for all life on earth.

Dealing with the global impact of increasing CO₂ concentrations in the atmosphere will require the development of new technology options. One of these is the use of both terrestrial plants and marine algal species to use solar energy in a photosynthesis cycle to remove carbon dioxide from the atmosphere. Such technology is known, for example, from Bernemann, J. R. (2003). Biofixation of CO₂ and Greenhouse Gas Abatement with Microalgae—Technology Roadmap, US Department of Energy, National Energy Technology Laboratory. The resultant bio-mass can then be used for production of sustainable fuels or for high value crops and products where the atmospheric CO₂ has been sequestrated into solid form.

Algal species are overall sixty times more efficient in their conversion of CO₂ into bio-mass because of a combination of their higher photosynthetic efficiency and higher growth rates. Despite this increased efficiency, removal from the atmosphere of sufficiently large quantities of CO₂ will still require a substantial industrial scale up of an algal production system. This will need substantial amounts of physical real estate and very large quantities of water. WO2008/105649 suggests that the seas will provide readily available areas and to this end discloses the growth of algae in self-contained structures floating on the sea or on any body of water where the water required for the growth is contained within the floating structure by a sheet of plastic, rubber or any other suitable material impervious to water. FIG. 2 of the document discloses harvesting apparatus in the form of a base station connected by a pipe to the floating structures and having two settling ponds and an algae ‘nursery’.

The base station of WO2008/105649 is, however, shore based with the ‘nursery’ at or above the high tide mark such that water containing desirable species of algae can, at low tide, flow from the ‘nursery’ down a pipe to an offshore floating structure. Thus substantial amounts of shore real estate are still required. Moreover, even though the floating structures are offshore, the aforementioned tidal pumping mechanism will place severe limits on the length of the pipes connecting the structures to the base station. The floating structures will therefore remain in view from the shore, which may be unacceptable at many shoreline locations on aesthetic grounds.

The present invention has as an objective the provision of an improved repository for the water-based farming of biomass.

### DISCLOSURE OF INVENTION

According to the present invention, there is provided: a repository for the water-based farming of biomass, the repository being configured to be attachable to at least one other repository at six regularly-spaced locations on its periphery.

Both the repository and the at least one other repository provide a discrete location or base at which bio-mass can be grown. By virtue of being attachable to at least one other repository at six regularly-spaced locations on its periphery, repositories can be grouped together in a way that allows multi-directional degrees of freedom and the close packing of repositories into a group. Repositories attached to one another into groups may be easier to manage than individual repositories.

The repository may also be configured to be attachable to a mooring device which is held in a fixed location. In this way, the repository—along with any other repositories attached thereto—can be held in a fixed location, again making them easier to manage than if they were free to move. Since a single mooring device can potentially moor a large number of repositories in this way, the system is readily scalable.

The repository may be configured to be compliantly attachable to another repository or a mooring device, in particular in such a way as to only transmit horizontal loads but no vertical loads or bending moments. This allows a group of repositories to accommodate motion of the water in which the group is based.

The plan form of the repository may be such that its behaviour in waves is substantially independent of the direction of those waves relative to the repository. The repository may be hexagonal in plan form. It may be configured to be attachable to another repository or a mooring device at each of its six vertices.

The repository may be configured to at least partially float on the surface of the water in which it is to be based. In one embodiment, at least part of the periphery of the repository is buoyant in water.

The repository may be a receptacle for bio-mass and possibly a growth medium therefor. In one embodiment, the receptacle comprises a partition to separate the bio-mass from the water in which the receptacle is to be based. The partition may be impermeable to the water in which the receptacle is to be based. The partition may be flexible, allowing the motion of the water to be transmitted to the bio-mass, which may agitate the bio-mass to promote faster growth. The flexible partition may be a flexible membrane.

The repository may comprise a peripheral structure that surrounds the flexible membrane. The peripheral structure may be deformable. It may be held in its plan shape by a stiffening structure. This may extend between opposite points on the peripheral structure. it may comprise a space frame extending between opposed vertices of the hexagonal plan form.

The receptacle may be of hexagonal plan form, with each side of the hexagon being about 25 metres in length. It may have a draft of about 2 metres. The hexagonal form promotes agitation (sloshing) of the biomass in the receptacle.

The receptacle periphery may be configured to overhang the surface of the bio-mass in the receptacle so as to prevent or at least reduce spillage of bio-mass out of the receptacle.

The present invention also provides a mooring device configured to be attachable to at least one repository as specified above. The mooring device may be buoyant in water. The mooring device may be hexagonal in plan form. It may be configured to be attachable to a repository at each of its six vertices.

A plurality of repositories may be combined with harvesting apparatus to form a bio-mass farming system, the harvesting apparatus being configured to be located in water and, at any one time, to harvest bio-mass from at least one but not all of the plurality of repositories. Since both repositories and harvesting apparatus are located in water, i.e. surrounded by a body of water (such as an ocean, sea or lake), such a system is not located on a shore. As such, it does not use either land or land-based water sources. Rather, it can be located anywhere on the ocean, sea or lake and advantageously out of sight from land.

Such a system may also be more cost-effective to implement since the harvesting apparatus, which is the most complex and expensive element of the system, only needs capacity sufficient to process a fraction of the total number of repositories—typically only those repositories that are ready for harvesting—at any one time. As a result, it may be possible to construct, at acceptable cost, large systems able to produce large volume rates of bio-mass. This bio-mass can serve as a means of sequestering atmospheric and dissolved seawater carbon dioxide and of producing high value bio-plastics, bio fuels and other farmed products.

The bio-mass may include macro- or micro-algal species. In one embodiment, the bio-mass are micro-algae having a growth cycle (from seeding to harvest) of three to seven days.

Like the repositories, the harvesting apparatus may also be configured to float or to be mounted on the floor of the body of water, e.g. on the ocean, sea or lake bed.

The repositories and harvesting apparatus may be moveable relative to one another in a horizontal direction, parallel to the surface of the water. Where the repositories are configured to float, such movement will typically be on the surface of the water

The harvesting apparatus may be configured to be fixed, with the repositories being moveable to and from the harvesting apparatus. Alternatively, the harvesting apparatus may be configured to be moveable to and from each repository.

A plurality of repositories may be processed by the harvesting apparatus at any one time. Attachment between repositories facilitates the transport of such a plurality of repositories to the harvesting apparatus. In one embodiment, the repositories arranged in a line for processing by the harvesting apparatus.

The system may comprise multiple separate groups of mutually-attached repositories. Each group may be moored by a single mooring device. In one embodiment, the system comprises three separate groups each comprising at least one hundred repositories of hexagonal plan form, each side of the hexagon being about 25 metres in length, each repository having a draft of about 2 metres.

The repository may also be employed in a method of bio-mass farming comprising the steps of: providing a plurality of water-based repositories for growing bio-mass; providing water-based harvesting apparatus; and harvesting bio-mass from at least one of the repositories while simultaneously leaving other repositories unharvested;

The method may also comprise the step of seeding at least one repository with micro-algae having a growth cycle (from seeding to harvest) of up to seven days, in particular three to seven days.

The method may also comprise the step of providing multiple separate groups of mutually-attached repositories.

The step of harvesting bio-mass may comprise detaching one or more repositories from a group and moving them to the harvesting apparatus. The repositories to be detached and removed may be attached together, in particular so as to form a line of repositories.

The present invention also provides a floating pond consisting of a buoyant framework with at least one floating member, a liner attached to the framework, a culture, and a mooring system.

The framework may be constructed with at least two floating members. At least two of said floating members may be longitudinal members. The framework may further comprise at least one transverse member, and the transverse member may attach to the longitudinal members. The framework may comprise at least two transverse members. The longitudinal members may be parallel, with said transverse members being perpendicular thereto. The transverse member may be constructed of a buoyant substructure. The buoyant substructure may be arc shaped.

The floating member may be mounted with at least one circumferential band. The circumferential band may include at least one mechanical element such as a point of attachment.

The floating member may be used as a mounting base. The floating member may be a composite constructed of at least two parallel members. At least one of the parallel members may be mounted with at least one circumferential band. At least one of the parallel members may be used as a mounting base. The circumferential band may include at least one mechanical element such as a point of attachment. The floating member may be constructed with at least one tubular element or at least two tubular elements.

The culture may be separated from surrounding water by the liner. The culture may be harvested by removing at least part of said culture from said floating pond. The culture may be maintained by adding water and nutrients to the floating pond. The culture may be an algae culture.

The mooring system may comprise at least one mooring line directly anchoring the framework to subsurface. Alternatively, in addition, the mooring system may comprise at least one mooring line connecting said framework to a buoy that is anchored to subsurface.

The floating pond may be submersible.

### BRIEF DESCRIPTION OF DRAWINGS

Embodiments of the invention will now be described in the accompanying drawings in which:

FIG. 1 is an aerial view of a typical farming system comprising three groups or ‘clusters’ of up to 90 repositories or ‘pods’.

FIG. 2 is a closer view of one of the pod clusters with a central pod that is used to moor the pod to the sea bed. Each individual pod is connected to adjacent pods to form an interlinked flexible ‘mat’ structure

FIG. 3 is an underwater view of a pod cluster with the central pod moored to the ocean or sea bed by mooring wires.

FIG. 4 is a three way perspective view of an individual pod showing its detailed structure.

FIG. 5 shows a typical production facility where a string of pods is being harvested and re-seeded prior to being returned to one of the pod clusters.

FIG. 6 shows an interior view of the facility with de-watering and drying equipment for the bio-mass prior to its despatch from the facility.

### DETAILED DESCRIPTION OF SPECIFIC EMBODIMENTS

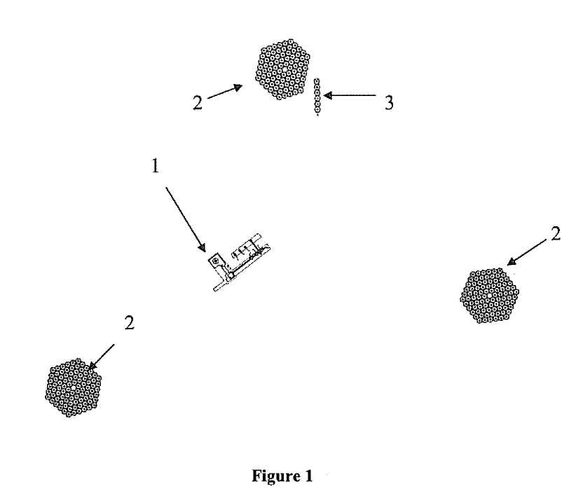

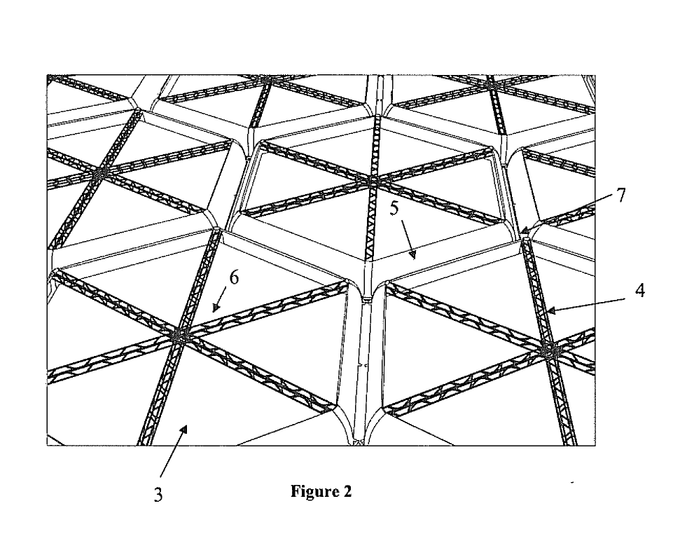

FIG. 1 shows a bio-mass farming system comprising a central processing facility, 1, surrounded by a multiplicity of groups (or ‘clusters’) of floating repositories or ‘pods’ 3. FIG. 1 shows a typical embodiment with three clusters 2 of ninety pods each. The pods are interlocked to form compliant floating ‘mats’ which serve as receptacles for the bio-mass—see FIG. 2. The system has means, in this embodiment tugs are used, to periodically transport strings of pods to the production facility for harvesting and then returning harvested, re-seeded pods back to their clusters.

The repeatable, inter-connectable bio-mass production pods are used as growth receptacles for micro-algal and macro-algal species. A particular embodiment designed to grow micro-algae is described here.

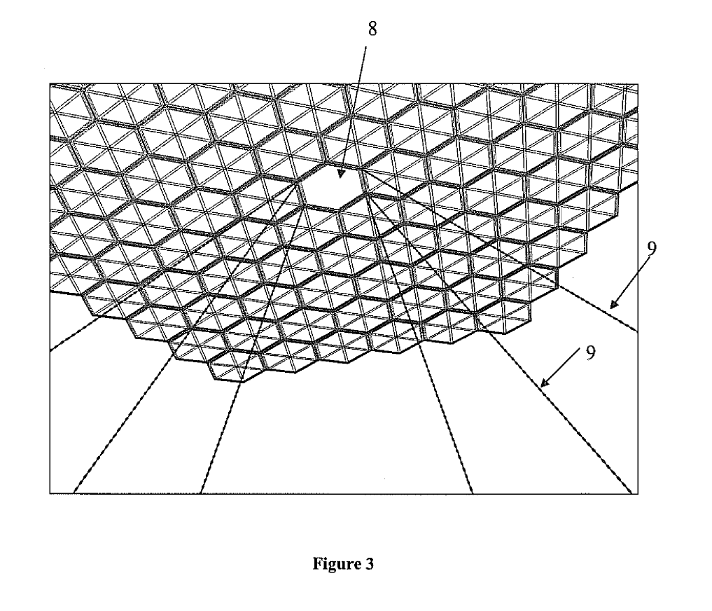

FIGS. 2 and 3 show different views of the pods 3 in their cluster. In one realisation, the pods are structures, hexagonal in plan form, with each side being about 25 m in length, with a draft of about 2 m and a total height of about 5 m. The purpose of the pods is to house and serve as a receptacle for a volume of growth medium within which a micro-algal species mix can grow rapidly. The pods (see FIG. 2) are made up as structures stiffened by space frames made up of members 4, which hold up a buoyant perimeter structure 5. The stiffening structure extends between diametrically opposite points on the peripheral structure: in the embodiment shown, the struts 4 extend between opposed vertices of the hexagonal plan form.

The perimeter structure surrounds an impermeable flexible membrane or skin 6 which makes up the bottom and side boundary of the pods within which the bio-mass is contained. A flexible membrane is typically not self-supporting and the overall structure maintains its hexagonal shape by the stiffening from the space frames and a difference in water pressure across the membrane obtained by a higher level of water inside the pod than outside.

Each pod is able to deform in ocean waves and thus shed the resultant internal structural loads. The pod sides are slightly deformable whilst the pod bottom is fully deformable. The compliant motion of the pod bottom in ocean waves will also serve to agitate the bio-mass such as algae within it to promote faster growth. Collisions between pods in a raft due to wave and current action will also agitate the biomass. The substantially circular shape of each hexagonal pod ensures that the pod's behaviour in waves from any direction is similar.

Each pod has devices on its vertices, such as in 7, to enable it to be compliantly connected to other pods. These six, regularly-spaced locations on the periphery of the pod allow the pods to be co-located in a cluster and to be close packed. The particular hexagonal shape of the pods ensures that, when interlaced, they do not lose any plan area in the overall geometry, in contrast to truly circular pods. The pods have means for manual access to monitor and maintain its functions together with facilities for handling, towing, mooring and managing them.

FIG. 3 shows an underwater view of a pod cluster with the central pod, 8, moored to the sea bed by light weight mooring lines, 9, and anchors (not shown). This central dummy pod, 8, is fitted with catenary mooring equipment housed on it, all other pods being connected to this centrally moored pod through these vertex connections.

Advantageously, each individual pod cluster is moored on a very slack mooring geometry such that the large drift of the cluster will avoid any particular part of the underlying water and sea bed to be shaded for long periods. Moreover, the individual pod clusters may also be well separated (as shown in FIG. 1) such that any accidental releases of algae into the surrounding water are quickly diluted. By choosing locally-evolved algae, any such escape or spillage of the bio-material will not endanger the local environment and indeed may actually boost marine life by providing a food source that arises naturally in the local environment;

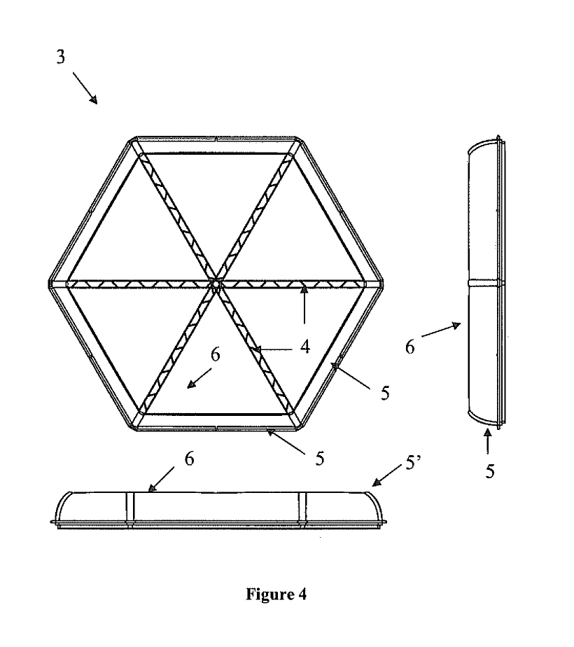

FIG. 4 shows a perspective three way view of one realisation of a pod 3. Whilst a standard pod may be hexagonal or other repeatable plan view in shape, its internal configuration may be custom designed for the bio-mass being cultured. As indicated at 5′, the inwardly-facing wall of the peripheral structure 5 may also be configured to overhang the surface of the bio-mass so as to prevent or at least reduce spillage of bio-mass out of the pod.

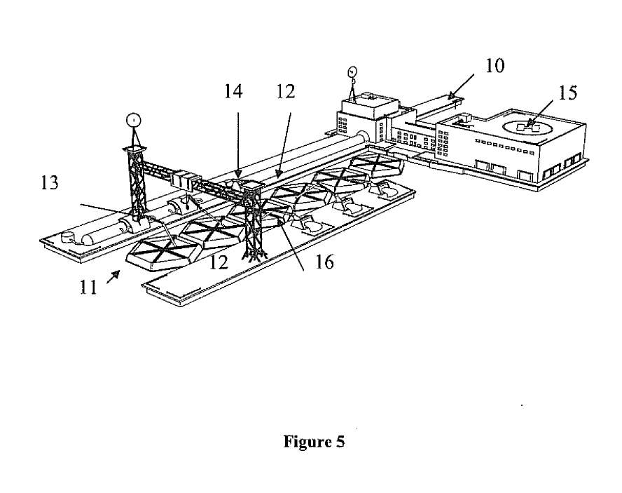

The farming system is based around a central processing facility shown in FIG. 5. This is a floating or bottom mounted structure, 10, with several specific physical attributes. The facility has a shaped channel, 11, or quays, 12, at which the standard pods would be brought in. In the embodiment shown, six pods can be accommodated at any one time, which is a small fraction—and certainly not all—of the total (three clusters of ninety pods) number of repositories in the system. For micro-algae, the harvesting and reseeding is done by fluid-handling machinery combined with filters, separators and de-watering equipment located on the central processing facility.

For micro-algae growth pods, the treatment channels, 11, on the processing facility have suction arms, 13, and pumping machinery, 14, to ‘harvest’ the algae mass and transport the contents to a processing plant, 15, on the facility. Further along the channels, other treatment arms, 16, have cleaning, re-seeding and measurement functions so that each algal pod can be configured to re-start its growth cycle after it is returned to its cluster.

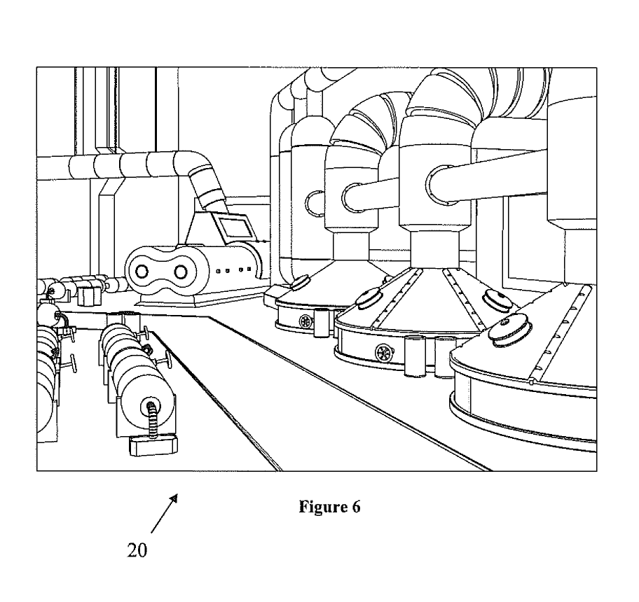

FIG. 6 shows an internal view of a typical facility that may fulfil this function. The facility is installed with machinery to process the algal bio-mass ready for transportation to end users. For the micro-algal bio-mass this entails filtering, de-watering, treating, drying and compressing the bio-mass, which can then be packaged or baled ready for transportation by ship from the processing facility.

A similar, but different machinery scheme (not illustrated here) may be required to handle such processed macro algae. The facility may need a quay side and automated equipment to load the ‘bales’ on to ships for transportation. The facility may also need to house a biological culture and measurement laboratory together with office, accommodation and maintenance facilities.

A part of the system design and engineering will be based around the three to seven day growth cycles of the micro-algae. Thus, after seeding, an algae pod will be placed in its cluster. Within three to seven days of this placing the pod will need to return to the central facility to be harvested, cleaned, re-seeded and returned to its cluster.

A factor in achieving this cycle for as many pods a day as possible will be the handling systems used. Having the pods floating on water makes it easy and cost-effective to move large volumes of bio-mass around. One embodiment of this is to use light diesel tugs handling trains of up to six pods to and from the central facility.

The speed at which the pods can be moved combined with the speed of harvesting and re-seeding will determine the efficiency and effectiveness of this process. Consider, for example, a facility comprising very many pods, each pod being hexagonal in plan area, of 25 metre per side and having a total plan area of 1623 square metres. With an algal growth medium depth of 0.5 metres, the pod will have a total growth medium volume of 1847 cubic metres. Such a facility will have a total plan area of several square kilometres. By processing 174 pods per day through the harvesting facility, a combined bio-mass and growth medium volume of 321,469 cubic metres per day can be achieved.

1.-23. (canceled)

24. A repository for the water-based farming of micro-algal bio-mass in a water growth medium, wherein the repository is configured to be attachable to at least one other repository at six regularly-spaced locations on its periphery such that collisions between the repositories due to wave and current action will agitate the micro-algal bio-mass.

25. A repository for the water-based farming of micro-algal bio-mass in a water growth medium according to claim 24, wherein the plan form of the repository is such that its behaviour in waves is substantially independent of the direction of those waves relative to the repository.

26. A repository for the water-based farming of micro-algal bio-mass in a water growth medium according to claim 25, wherein the repository is hexagonal in plan form.

27. A repository for the water-based farming of micro-algal bio-mass in a water growth medium according to claim 26, wherein the repository is configured to be attachable to another repository at each of its six vertices.

28. A repository for the water-based farming of macro-algal bio-mass in a water growth medium according to claim 24, wherein the repository is a receptacle for containing the bio-mass and comprises a partition to separate the biomass from the water in which the receptacle is to be based, wherein the partition is flexible, allowing the motion of the water in which the receptacle is to be based to be transmitted to the bio-mass, thereby agitating the bio-mass to promote faster growth.

29. A repository for the water-based farming of micro-algal bio-mass in a water growth medium according to claim 28, wherein the partition is a flexible membrane, the repository comprising a peripheral structure that surrounds said flexible membrane.

30. A repository for the water-based farming of macro-algal bio-mass in a water growth medium according to claim 29, wherein said peripheral structure is deformable.

31. A repository for the water-based farming of macro-algal bio-mass in a water growth medium according to claim 30, wherein said peripheral structure is held in its plan shape by a stiffening structure.

32. A repository for the water-based farming of macro-algal bio-mass in a water growth medium according to claim 31, wherein said stiffening structure extends between opposite points on said peripheral structure.

33. A repository for the water-based farming of macro-algal bio-mass in a water growth medium according to claim 32 wherein the repository is hexagonal in plan form and wherein the stiffening structure comprises a space frame extending between opposed vertices of the hexagonal plan form.

34. A repository for the water-based farming of macro-algal bio-mass in a water growth medium according to claim 28, wherein the repository is a receptacle of hexagonal plan form, with each side of the hexagon being about 25 metres in length, and having a draft of about 2 metres.

35. A repository for the water-based farming of macro-algal bio-mass in a water growth medium according to claim 29, wherein the peripheral structure is configured to overhang the surface of the bio-mass in the receptacle.

36. A mooring device configured to be attachable to six repositories according to claim 24.

37. A mooring device according to claim 36, wherein the device is buoyant in water.

38. A mooring device according to claim 37, wherein the device is hexagonal in plan form and is configured to be attachable to a repository at each of its six vertices.
